(19) United States
(12) Patent Application Publication
(10) Pub. No.: US 2006/0292679 A1
(43) Pub. Date: Dec. 28, 2006

(54) Beer containing monascus-derived pigments

(76) Inventors: Jerome Pellaud, Dion-Valmont (BE); James W. Kerr, London (CA)

Correspondence Address: James W. Kerr;InBev S.A., 303 Richmond Street, London, ON, N6B 2H8, CA

(21) Appl. No.: 11/166,183

(22) Filed: Jun. 27, 2005

(51) Int. Cl. C12P 7/14; C12P 1/02

(52) U.S. Cl. 435/162; 435/171; 424/725; 424/439

(57) ABSTRACT

Beers and processes for the production thereof are provided in accordance with the present invention, in which Monascus—derived pigments are employed as natural (i.e. non-synthetic) colorants.

### FIELD OF THE INVENTION

The present invention relates to beer contain food colorant pigments derived from Monascus sp., and to various processes related thereto.

### BACKGROUND OF THE INVENTION

According to Fabre et al, (in Production and Food applications of the Red Pigments of Monascus ruber—Journal of Food Science 58(5) 1993-p 1099-1110), food coloration although typically devoid of nutritional value, is important factor in improved food presentation and hence a contributing factor to its enjoyment. Accordingly, the development of comestibles that lend themselves to attractive presentations has and continues to be an important objective in the commercial food industry.

For over a century and a half, the use of synthetic food colors was been widely practiced—with costs, color control/reproducibility, and product stability being amongst the driving forces behind their popularity.

Over the last 10 to 15 years, however, regulatory approval for some of the mainstream synthetic food colorants has been withdrawn. Synthetic colorants are viewed with suspicion by many among the consuming public, and regulators have found cause for those concerns in synthetic colorants that have proven to be potential carcinogens or allergens.

The demise of the “synthetic option” however, has not done away with the desire to craft visually appealing food products—and as a consequence the food industry has turned its attention to natural sources of food colorants. In particular much attention has been paid to fungal pigments. For example, Wong, (PhD thesis—Antibiotic and pigment production by Monascus purpureus, 1982, University of Georgia, Athens), noted that more than 50 patents had been recently issued concerning various uses of Monascus pigments.

Colorants produced by the Monascus genus of fungi are well known in the Orient. Traditionally grown on rice or bread in semi-solid or solid fermentations, Monascus species produce red orange and yellow pigments—and according to Went, (1895 Monascus purpureus—Une nouvelle Thelebolee—Le champignon de L'Ang-Quac. Ann. Sci, Nat. Bot, 8(1): 1-17), have been in use for hundreds of years as a general food colorant and medicinal agents.

Fabre et al, (supra) examined the use Monascus ruber pigments as natural colorants for meat applications. They concluded that these pigments were superior to nitrite salts in these applications and that the color remained stable (95%) even after three months of storage of colored meat product. However, Fabre et al also noted that in solutions containing these pigments, (and in particular the red pigments), where very unstable at elevated temperatures and acid pH. Wong and Koehler (Production of red water soluble Monascus pigments J. Food Sci, 48: 1200-1203-1983), corroborated these results—although work by Francis (Lesser known food colorants, Food Technol. 41 (4): 62-68), found the opposite.

On the basis of the work done by Fabre et al, and confirmed by Wong and Koehler—persons skilled in the art of brewing would have reason to believe that Monascus sp. pigments would not be suitable for use in brewing owing in part to the acidity of beer and in part to the heating employed in the kettle boiling and pasteurization steps used in most brewing operations.

Moreover, brewers already had to hand the roasted malts that are traditionally used to impact color to their products. These malt colorants are produced by Maillard reactions during roasting and unlike the instability attributed to the Monascus pigments, these roasted malt colors have proven to be stable and well suited to the beer application. Accordingly, brewers have little incentive to adopt Monascus pigments, especially given the discouraging prospects follow from Fabre et al and Wong and Koehler's teachings that lead away from such a potential application.

Moreover, the pigment production by the Monascus genus is associated with the co-production of hydroxymethylglutaryl coenzyme A reductase inhibitors which interfere in the sterol metabolism of Saccharomyces cerevisiae—and this in turn compromises the yeast cells viability, (Lorenz and Parks, Effects of Lovastatin (Mevinolin on Sterol Levels and on Activity of Azoles in Saccharomyces cerevisiae, Antimicrobial Agents and Chemotherapy, September 1990, p. 1660-1665). While this finding attends substantial advantages in treating fungal infections with antimicrobial azoles—anything that compromises the yeasts functioning in the brewing process is viewed, a priori, as undesirable.

Accordingly, while natural Monascus sp. pigments may be desirable, advantageous and even necessary as alternatives to synthetic pigments over a broad range of food applications, there is reason to perceive its application in brewing as fundamentally different—and indeed as an exception to their more general utility outside of that application.

### SUMMARY OF THE INVENTION

In accordance with a broad aspect of the present invention, and notwithstanding the countervailing teachings of the prior art, there is provided a beer comprising an at least one food colorant derived from pigment-producing Monascus sp. and a variety of processes for the production thereof. In accordance with the invention, the colorant in question may be produced either exogenously from, or endogenously during, the brewing process.

Endogenous production of the colorant may be:

during mashing, by way of a selected one or combination of both:
culturing grain and/or cereal adjunct with pigment-producing Monascus sp under pigment-producing conditions; or
culturing malt with pigment-producing Monascus sp. under pigment-producing conditions.

Alternatively, or in addition if desired, the colorant can be produced exogenously:
in a malting process wherein pigment-producing Monascus sp. are employed as a starter culture under pigment-producing conditions; and/or,
in a syrup producing process wherein:
   the syrup is a malt syrup produced from a malt saccharification process including culturing said malt with a pigment-producing Monascus sp. under pigment-producing conditions; and/or,
   the syrup is an adjunct syrup produced from an adjunct saccharification process including culturing said adjunct with a pigment-producing Monascus sp. under pigment-producing conditions; and/or,
as an exogenous extract from a cultured substrate, cultured using a pigment-producing Monascus sp. under pigment-producing conditions; and/or,
as an exogenously cultured substrate cultured using pigment-producing Monascus sp. under pigment-producing conditions.

The present invention also relates to the various processes of producing the forgoing, including the steps of introducing the Monascus—derived colorant to the brewing process, in each case.

### DETAILED DESCRIPTION OF THE INVENTION:

In accordance with a general aspect of the present invention there is provided a beer comprising an at least one food colorant derived from pigment-producing Monascus sp.

The red and yellow pigments of Monascus purpureus, such as monascorubin and monascin, have been purified and extensively studied (Fielding et al., 1961, J Chem Soc, 4579-4589). The culture conditions and its effect on pigmentation of Monascus purpureus have also been studied (Broder et al., 1980, J Food Sci, 45:567-469).

The pigments produced by Monascus species traditionally grown on rice in the Orient are orange and relatively insoluble in water, but readily react with compounds containing amino groups to form water soluble red colorants. The orange pigment is a mixture of monascorubrin and rubropunctatin, whose structures were elucidated by B. C. Fielding et al., Tetrahedron Letters No. 5, 24-7 (1960) and Kumasaki et al., Tetrahedron, 18, 1171 (1962), and which differ in the former having a 7-carbon ketonic group and the latter having a 5-carbon ketonic group.

Commercial production of precursor pigment requires development of a suitable fermentation procedure, which has been the subject of many reports in recent years. Shepherd et al., U.S. Pat. No. 4,145,254, made an important advance by using a two-stage process in which the microorganism first was cultivated at pH 4-7 in a growth-promoting medium, then was transferred to a second medium at pH 2-4 to stimulate precursor pigment production. The low pH did not interfere with precursor pigment production but inhibited its subsequent reaction with amino groups of proteins and/or ammonium ions in the medium. The result was the exclusive production of orange precursor pigment as a colorant. As another example U.S. Pat. No. 4,442,209 claims to increase precursor pigment formation by cultivating a Monascus species in a medium containing maltitol.

A selected one or a mixture of Monascus species and/or strains can be employed in the practice of the present invention such as Monascus purpureus Went; Monascus rubervan Tieghem; Monascus Fuliginosus Sato; Monascus Pilosus Sato; Monascus albidus Sato; Monascus pubigerus Sato; Monascus paxii Lingelsheim; and/or Monascus anka

### EXAMPLE 1

In accordance with one aspect of the present invention the beer and the process whereby it is produced involves the colorant being produced endogenously during the brewing process.

A conventional brewing process includes the steps known as mashing, wort separation, kettle boiling, fermentation, maturation etc. During the mashing process, malt and cereal/grain adjuncts are treated to produce a fermentable wort. In accordance with this aspect of the present invention, the any one or more or a combination of malt and cereal/grain adjuncts are cultured with a pigment-producing Monascus sp under pigment producing conditions. The resulting pigment production is then carried forward as a colorant through the balance of the brewing process and into the final beer product.

Barley and other malts are within the contemplated practice of this aspect of the present invention, as are corn, rice and other adjuncts.

General culture conditions that are also suitable for the purposes of the present invention are variously described in U.S. Pat. Nos. 4,418,080; 3,911,141; 4,031,250; 4,145,254; and 4,418,081. Note in particular, the addition of nutrient solution to help advance the Monascus culture. Once the Monascus culturing has advanced to a desired degree, the mashing process is completed in order to produce the wort—and the process of the present invention then proceeds by extracting the now pigmented wort from the spent grains, boiling and fermenting the wort with brewer's yeast and finishing the beer.

A strain of a microorganism of the genus Monascus which produces pigment of the monascorubrin or rubropunctatin type may be used for carrying out the process according to the subject invention. An inoculum of the strain selected may be prepared from a reconstituted, freeze dried culture, or from a culture on a solid or liquid medium by any method known in the art.

In general the method of the subject invention involves preparing the malt and/or cereal/grain substrate to be inoculated in a moist or aqueous fashion. The substrate in the mash mixer or cooker or other mashing vessel as the case may be, then inoculated with the Monascus mold so that the viable organism is evenly distributed throughout the growth medium and then allowed to ferment unhindered until completion of its growth cycle, as monitored by the completion of coloration of the substrate. The moisture level is maintained at approximately 30% throughout the growth cycle of the organism by addition of water as necessary.

As mentioned above, a nutrient solution comprising, for example, a mixture of magnesium sulfate, sodium nitrate, and potassium phosphate and blending this nutrient solution with the substrate. For maximum color levels, the concentration of the nutrient solution should be such as to permit optimum growth of the mold on the substrate, as known in the art. The nutrient solution may include amino acids, sugars, starches, protein hydrolysates, molasses, casein, yeast extracts and other ingredients as known in the art.

Note that the Monascus sp., in addition to producing the desired pigments, also produces various saccharification enzymes that otherwise contribute to the desired outcomes of the mashing process by improving conversion of carbohydrates to fermentable sugars useful in the subsequent yeast fermentation step. In addition, Monascus cultures have an antibacterial effect that can aid in avoiding infections during this early stage of the brewing process.

### EXAMPLE 2

In accordance with other aspects of the present invention, the colorant is produced exogenously from the brewing process.

### EXAMPLE 2a

In accordance with one such aspect, the colorant is produced exogenously in a malting process wherein pigment-producing Monascus sp. are employed as a starter culture under pigment-producing conditions.

According to this aspect of the present invention, there is provided a process for treating barley, wheat or sorghum, comprising wetting the grain/cereal in question, to increase its moisture content sufficiently so that on being held for a time and temperature it will germinate—and wherein a pigment-producing Monascus starter culture is mixed with the grain with before, during or after the wetting of the cereal/grain.

The starter culture can be introduced before or during the various germination or steeping stages. The pitching rates of the culture vary depending on the conditions of the malting process.

The grain/cereal is wetted and mixed with a starter culture which may be combined with the seeds either before or after wetting. In this aspect, the grain, starter culture are combined before, during or after the wetting the grain and the combination is held at a temperature of at least about 5.degree. C. and not more than about 30.degree. C., preferably between about 10.degree. C. to about 20.degree. C. The wetted or moistened grain and culture combination is held for a time and temperature until the grain has a moisture content of at least about 20 weight percent and are preferably held at a moisture content of between about 20 to about 60 weight percent, preferably from about 38 to about 47 weight percent, so that the grain germinates for about 2 to about 7 days, preferably about 3 to about 6 days, at a temperature of from about 10.degree. to about 30.degree. C., preferably from 14.degree. to about 21.degree. C. The germinated grain is then dried, typically at a temperature of from about 40.degree. to about 150.degree. C., preferably between about 45.degree. and 85.degree. C. until the dried pigmented malt has a moisture content of from about 2 to about 15 weight percent moisture, preferably from about 3 to about 7 weight percent moisture.

The malt is then used in a brewing process wherein the pigmented malt is mashed to form a pigmented wort in which the Monascus colorant is then carried through the balance of the brewing process and into the finished beer.

### EXAMPLE 2b

In accordance with yet another aspect of the presentation, the colorant is produced exogenously in a syrup producing process. More particularly, the syrup is a malt and/or fermentable adjunct syrup produced from a malt/adjunct saccharification process including culturing said malt with a pigment-producing Monascus sp. under pigment-producing conditions.

A preferably at least partially gelatinized starch substrate such as steamed rice is inoculated with pigment-producing Monascus sp. This is then mixed with water and heated for a period of time to initiate saccharification, and then the liquid syrup is separated from the solid residue. The liquid syrup recovered is a saccharified pigment-containing syrup preferably in the range of 15-30.degree. Brix;

The temperature and length of time for saccharification are adjustable. The saccharification can be conducted at 30.degree. C. for 12-24 hours, but should be preferably performed at a higher temperature (above 50.degree. C.) to shorten the saccharification time to 2-6 hours, depending upon the saccharifying power of the selected Monascus sp. and the desired sugar content of the saccharified syrup.

The syrup together with the colorant that it contains is then employed in known manner as a liquid adjunct to the beer brewing process—to be fermented by the brewers yeast and carry the pigment forward through the balance of the brewing process to produce a beer having a colorant therein.

### EXAMPLE 2c

In a further aspect according to the present invention a beer is produced in the course of a beer brewing process in which the colorant in question is exogenously produced as an extract from a cultured substrate, cultured using a pigment-producing Monascus sp. under pigment-producing conditions.

The production of a cultured liquid substrate has already been disclosed herein and is generally well understood in the microbiological arts in any case. Having been fermented or cultured, the fermentation liquid can be purified by various conventional techniques, e.g., the ionic exchange method as taught in IP 07216248, the method by controlling the solvent and pH values as taught in JP 61281158, and the ethanol purification methods used in JP 54033535, 53069887, and 52034985, or, the method taught in JP 03254686.

Thereafter, and in accordance with the practice of the present invention, the pigment extract can be added during the brewing process to produce a beer having the desired colorant present therein.

### EXAMPLE 2d

Lastly, in accordance with a final aspect of the present invention, a beer is produced in accordance with a brewing process in which the colorant is exogenously produced as a cultured substrate cultured using pigment-producing Monascus sp. under pigment-producing conditions.

The carbon source in the cultured substrate can be rice (polished long-grain non-glutinous rice, polished round-grain non-glutinous rice, polished glutinous rice, red rice, and black rice), millet, barley, wheat, or corn. Additionally sugar and substances containing sugar can be used. Organic compounds such as glycerine and glyceride can also be used in the media preparations. For each 100 g of polished round-grained nonglutinous rice, 30-80 ml of Monascus sp. culture medium are added. The culture media's carbon source is selected from the group consisting of cereals, sugar, and organic compounds, the source of nitrogen is selected form the group consisting of beans (e.g. soya bean powder, pressed soybean cake), or peanut powder (or pressed peanut cake), peptone, rice extract powder, thick beef juice, silkworm chrysalis powder, or inorganic salts (e.g. NH.sub.4 NO.sub.3, etc.), and a source of phosphorous can also be added, such as inorganic salts (e.g. KH.sub.2 PO.sub.4, K.sub.2 HPO.sub.4, etc). Other inorganic salts can also be added, such as MgSO.sub.4 or FeCl.sub.2.

Approximately 40-80 ml of the mixture is added to each 100 g of rice, the pH is maintained at 3-8, and it is sterilized in steam at 121.degree. C.

Generally, the pH is adjusted to 3.0-5.0, and the mixture is steam sterilized (121.degree. C.). The mixture is cooled to 40.degree. C. and the rice is inoculated with the a Monascus strain and cultured at 15-35.degree. C. for 9 days. Fermentation of the rice mixture is preferably carried out at a temperature of 15-35.degree. C., most preferably 20-28.degree. C., for over 4 days, most preferably 9 days or more, until the formation of desired pigmentation is realized. Any one of a number of methods of fermentation, well known to one of skill in the art, can be used.

At the end of the fermentation process, the fermentation broth is drained and discarded, while the solid residue is sterilized by heat (for example, by high pressure steam). For example, the fermentation product is sterilized at a temperature of 69-121.degree. C., and dried.

This dried product can be ground for addition into the brewing process—into which it can be added so that the pigment that it contains is passed through the process and into the final beer as a colorant therein. To the extent that this solid residue contains fermentable sugars, it is preferred that the material be added to the brewing process while some fermentation activity is still ongoing.

### EXAMPLE 2e

### A Preferred Embodiment of the Present Invention

In accordance with this practice of this aspect of the present invention, a malt beer having a mash bill that ordinarily employed 80% malt and 20% corn as a cereal grain adjunct was prepared, substituting 10% of the maize adjunct with an equal amount of a Monascus purpureus fermented, red-pigmented rice.

The Monascus—cultured rice was supplied by Jiangr Bio-Technology, Co. Ltd., Yiuwu City, Zhejiang Province (China), according to the following specifications:
moisture (6.3%)
friability 97%
extract (% as is) 68%
extract (% dry mat) 74%
total proteins (% dry mat) 14.91
FAN (ppm) 116
beta-glucan (ppm) 12
fatty substances (g/100 g dry mat) 4.17
   The whole rice kernels had a 97% friability.

The rice kernels were employed whole, without grinding and were added to the cereal cooker in the same manner as the corn.

[image]

The corn and Monascus cultured rice were heated in the cereal cooker as indicated in the above graph, to a temperature of 100 degrees C., held at that temperature for 10 minutes and then allowed to cool to 63 degrees C., at which point the corn and rice were added back into the ongoing malt mashing process.

Extraction from the mash using a lauter tun produced wort at a 92% yield rate. The wort analysis appears in the following table:

| | | |
|---|---|---|
| Original Extract | ° PI | 15.11 |
| Apparent Extract | ° PI | 15.11 |
| Real extract | g/100 g | 15.11 |
| Alcohol by Weight | g/100 g | 0 |
| | ml/100 | |
| Alcohol by Volume | ml | 0 |
| pH | | 5.1 |
| Limit Extract | ° PI | 2.35 |
| Apparent-Limit | ° PI | 12.76 |
| Extract | | |
| Bitterness | BU | 28.5 |
| Polyphenols | mg/l | 340 |
| Nitrogen, N | mg/l | 1237 |
| Free Amino Nitrogen | mg/l | 265 |
| Beta Glucans | mg/l | 113 |
| Dextrin | | 740 |
| Calcium | ppm | 52 |
| Linoleic Acid | ppm | 5.38 |
| Linolenic Acid | ppm | 0.64 |
| Oleic Acid | ppm | 1.43 |
| Palmitic Acid | ppm | 4.17 |
| Stearic Acid | ppm | 0.53 |
| Total Fatty Acids | ppm | 12.14 |
| Fructose | g/100 ml | 0.17 |
| Glucose | g/100 ml | 1.5 |
| Maltose | g/100 ml | 6.66 |
| Maltotriose | g/100 ml | 1.71 |
| Saccharose | g/100 ml | 0.26 |
| Total Sugar | g/100 ml | 10.3 |
| Zn | ppb | 112 |
| Viscosity | Mpa · s | 2.25 |

The wort was then boiled in a brewing kettle, cooled and finally pitched with brewer's yeast to ferment over a period of 9 days at a temperature of about 13 degrees C. The beer was brewed at 15.4 degrees Plato and was not diluted.

Following fermentation, the beer had a strong orange coloration and a nutty—bread like aroma associated with the cultured rice product.

1. A beer comprising an at least one food colorant derived from pigment-producing Monascus sp.

2. The beer according to claim 1, wherein said colorant is produced exogenously from the brewing process.

3. The beer according to claim 1, wherein said colorant is produced endogenously during the brewing process.

4. The beer according to claim 3, wherein said colorant is produced endogenously during mashing, by culturing grain and/or cereal adjunct with pigment-producing Monascus sp under pigment-producing conditions.

5. The beer according to claim 3, wherein said colorant is produced endogenously during mashing, by culturing malt with pigment-producing Monascus sp. under pigment-producing conditions.

6. The beer according to claim 2, wherein said colorant is produced exogenously in a malting process wherein pigment-producing Monascus sp. are employed as a starter culture under pigment-producing conditions.

7. The beer according to claim 2, wherein said colorant is produced exogenously in a syrup producing process.

8. The beer according to claim 7, wherein said syrup is a malt syrup produced from a malt saccharification process including culturing said malt with a pigment-producing Monascus sp. under pigment-producing conditions.

9. The beer according to claim 7, wherein said syrup is an adjunct syrup produced from an adjunct saccharification process including culturing said adjunct with a pigment-producing Monascus sp. under pigment-producing conditions.

10. The beer according to claim 2, wherein said colorant is exogenously produced as an extract from a cultured substrate, cultured using a pigment-producing Monascus sp. under pigment-producing conditions.

11. The beer according to claim 2, wherein said colorant is exogenously produced as a cultured substrate cultured using pigment-producing Monascus sp. under pigment-producing conditions.

12. A brewing process according to any of the preceding claims.
